(19) United States
(12) Patent Application Publication
(10) Pub. No.: US 2026/0062716 A1
(43) Pub. Date: Mar. 5, 2026

(54) LIPID NANOPARTICLE (LNP) COMPOSITION OR FORMULATION FOR NUCLEIC ACID THERAPEUTICS

(71) Applicant: PopVax Private Limited, Mumbai (IN)

(72) Inventor: Sourav Chattopadhyay, West Bengal (IN)

(21) Appl. No.: 19/106,079

(22) PCT Filed: Aug. 22, 2023
(86) PCT No.: PCT/US2023/030809

§ 371 Date: Feb. 24, 2025

Related U.S. Application Data
(60) Provisional application No. 63/400,545, filed on Aug. 24, 2022.

(51) Int. Cl. C12N 15/88; A61K 9/51; A61K 39/00; A61K 39/02; A61K 39/12

(52) U.S. Cl. CPC C12N 15/88; A61K 9/5123; A61K 9/5161; A61K 39/02; A61K 39/12; A61K 2039/53; A61K 2039/55555

(57) ABSTRACT

The present disclosure relates generally to lipid nanoparticle compositions comprising a nucleic acid, an ionizable polymer, a cationic lipid, a phospholipid, a sterol, and a PEG-lipid. Further, the present disclosure relates generally to methods of treating or preventing a disease, comprising administering to a subject in need thereof a lipid nanoparticle composition described herein.

### RELATE APPLICATIONS

This application claims the benefit of priority to U.S. Provisional Patent Application Ser. No. 63/400,545, filed Aug. 24, 2022, the contents of which are hereby incorporated herein by reference in their entirety.

### FIELD OF THE INVENTION

The present disclosure relates to a lipid nanoparticle (LNP) composition or formulation for nucleic acid therapeutics.

### BACKGROUND

Small molecules and protein based drugs or therapeutics have been used since several decades. Nucleic acid based therapies are emerging as a promising class of drugs in the recent times. These drugs comprise a segment of either deoxyribonucleic acid (DNA) or ribonucleic acid (RNA). Delivering nucleic acid to the target cells has been a challenge due to lack of suitable nucleic acid delivery vectors. Viral vectors have been used for this purpose, but innate and adaptive immune responses to these vectors and their transgene products presents substantial hurdles to their wider use (Shirley, Jamie L. et al. Molecular Therapy (2020) 28: 709-722).

In the recent years lipid nanoparticles (LNPs) have received a lot of attention as delivery vehicle for nucleic acid therapeutics. Patisiran is the first FDA approved short interfering RNA (siRNA) therapeutic encapsulated in a lipid nanoparticle (Kulkarni, Jayesh A. et al. Nature Nanotechnology (2021) 16: 630-643). Lipid nanoparticles offers immense promise in delivering nucleic acid based therapeutics to the target cells. At least two messenger RNA (mRNA) based vaccines against COVID-19 disease, which uses lipid nanoparticles as delivery vehicle, have been commercialized, and several of them are in different stages of clinical trials (Vu, Mai N. et al. EBioMedicine (2021) 74: 103699).

The mRNA vaccine commercialized by Moderna (SPIKEVAX®) is shipped and delivered at −20° C., and the one from Pfizer (COMIRNATY®) at −70° C. (Fahrni, M. L., et al. Journal of Pharmaceutical Policy and Practice (2022) 15: 16).

Successful mass administration of such products across various geographies during health emergencies may not be feasible as these lipid nanoparticles based nucleic acid therapeutics have to be stored at frozen temperatures creating issues for many countries, especially developing countries, in their storage, transport, and last mile delivery due to lack of availability of cold chain facilities.

WO2022101469 describes a lyophilized formulation comprising sucrose and/or trehalose as an additional component to the lipid nanoparticle formulation to improve the stability. However, lyophilized formulations require an extra step of reconstitution before being administered and may be susceptible to medication errors (Lee, Young Hwa et al. Vaccines (Basel) (2021) 9(2): 117).

Therefore, there is a need for new lipid nanoparticle compositions for nucleic acid based therapeutics.

### SUMMARY

Accordingly, the present disclosure relates to a lipid nanoparticle composition comprising a nucleic acid, an ionizable polymer, a cationic lipid, a phospholipid, a sterol and a PEG-lipid.

The present disclosure is generally directed to a lipid nanoparticle composition comprising a nucleic acid, an ionizable polymer, an ionizable lipid, a phospholipid, a sterol and a PEG-lipid. In some embodiments, the nucleic acid is a DNA, an RNA or combination thereof. The RNA may be a messenger RNA (mRNA), a non-coding RNA (ncRNA) or combination thereof. The non-coding RNA (ncRNA) may be long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA) or ribosomal RNA (rRNA) or combination thereof.

In some embodiments, the ionizable polymer is present in an amount from 1 mol percent to 25 mol percent. The ionizable polymer may comprise a biocompatible polymer. The ionizable polymer may be a chitosan, a cellulose derivative, a poly-L-lysine, a poly-L-glutamic acid, and/or their derivatives or combination thereof. The chitosan, chitosan derivatives or combination thereof may be present in an amount from 1 mol percent to 25 mol percent. In some embodiments, the cellulose derivative is present in an amount from 1 mol percent to 25 mol percent. In some embodiments, the chitosan or its derivatives or combination thereof is present in an amount from 1 mol percent to 25 mol percent, 1 mol percent to 20 mol percent, or 1 mol percent to 15 mol percent.

In some embodiments, the cationic lipid is present in an amount from 25 mol percent to 50 mol percent. The cationic lipid may be selected from N,N-dioleyl-N,Ndimethylammonium chloride (DODAC); N-(2,3-dioleyloxy)propyl)-N,N,Ntrimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammoniumtrifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), N-(1,2-dimyristyloxyprop-3-yl)-N,N-dimethyl-N-hydroxyethyl ammonium bromide (DMRIE), 1,2-dilinoleyloxy-N,N-dimethylaminopropane (DLin-DMA), 3-dimethylamino-2-(cholest-5-en-3-beta-oxybutan-4-oxy)-1-(cis,cis-9,12-oc-tadecadienoxy)propane (Clin-DMA), 2-[5′-(cholest-5-en-3-beta-oxy)-3′-oxapentoxy)-3-dimethyl-1-(cis,cis-9′,12′-octadecadienoxy)propane (CpLin-DMA), 2,3-dilinoleoyloxy-N,N-dimethylpropylamine (DLin-DAP), 1,2-N,N′-dilinoleylcarbamyl-3-dimethylaminopropane (DLincarb-DAP), 1,2-Dilinoleoylcarbamyl-3-dimethylaminopropane (DLin-CDAP), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), heptadecan-9-yl 8-[2-hydroxyethyl-(6-oxo-6-undecoxyhexyl)amino]octanoate (SM-102), 6-[6-(2-hexyldecanoyloxy)hexyl-(4-hydroxybutyl)amino]hexyl 2-hexyldecanoate (ALC-0315), and nonyl 8-[(8-heptadecan-9-yloxy-8-oxooctyl)-(2-hydroxyethyl)amino]octanoate (SLP-0001).

In some embodiments, the cationic lipid comprises an ionizable lipid. The ionizable lipid may be present in an amount from 25 mol percent to 50 mol percent.

In some embodiments, the phospholipid is present in an amount from 2 mol percent to about 20 mol percent. The phospholipid may be selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), 1-myristoyl-2-stearoyl-sn-glycero-3-phosphocholine (MSPC), 1-palmitoyl-2-myristoyl-sn-glycero-3-phosphocholine (PMPC), 1-palmitoyl-2-stearoyl-sn-glycero-3-phosphocholine (PSPC), 1-stearoyl-2-myristoyl-sn-glycero-3-Phosphocholine (SMPC), 1-Stearoyl-2-palmitoyl-sn-glycero-3-phosphocholine (SPPC), 1-stearoyl-2-oleoyl-sn-glycero-3-phosphocholine (SOPC), 1-stearoyl-2-docosahexaenoyl-sn-glycero-3-phosphocholine (SDPC), sphingomyelin, and combinations thereof.

In some embodiments, the sterol is present in an amount from 30 mol percent to about 65 mol percent. The sterol may be cholesterol, sitosterol, fecosterol, ergosterol, campesterol, stigmasterol, 5α-cholestanol, 5β-coprostanol, cholesteryl-(2′-hydroxy)-ethyl ether, cholesteryl-(4′-hydroxy)-butyl ether, 6-ketocholestanol, 5α-cholestane, cholestenone, 5α-cholestanone, 5β-cholestanone, cholesteryl decanoate, or their derivatives.

In some embodiments, the PEG-lipid is present in an amount from 0.2 mol percent to about 2.0 mol percent. The PEG-lipid may be mPEG-dimyristoyl glycerol (mPEG-DMG), mPEG-N,N-ditetradecylacetamide (mPEG-DTA or ALC0159), mPEG-cholesterol (mPEG-CLS), mPEG-DSPE, mPEG-DMPE, mPEG-DPPE, mPEG-DLPE, mPEG-DOPE, mPEG-DPPC, mPEG-DSPC, 1,2-distearoyl-sn-glycero-3-phosphoethanolamine with conjugated methoxyl poly(ethylene glycol) (mPEG-DSPE), 1,2-dimyristoyl-rac-glycero-3-methoxypolyethylene glycol-2000 (DMG-PEG 2000), or mixtures thereof.

In some embodiments, the nucleic acid encodes an antigenic polypeptide. The antigenic polypeptide may be derived from an infectious agent, such as strains of viruses or strains of bacteria.

In some embodiments, the nucleic acid regulates or modulates cellular functions.

In some embodiments, the nucleic acid comprises at least one chemical modification.

In some aspects, provided herein is a nucleic acid vaccine or therapeutic comprising the lipid nanoparticle composition described herein.

In some aspects, provided herein is a method of treating or preventing a disease, comprising administering to a subject in need thereof the lipid nanoparticle composition described herein or the nucleic acid vaccine or therapeutic described herein. In some embodiments, the disease is cancer, an infectious disease or a disease and/or disorder ameliorated by humoral and/or cellular immune response.

In some aspects, provided herein is a method of preparing the lipid nanoparticle composition described herein, comprising mixing an aqueous phase comprising the nucleic acid and the ionizable polymer, and an organic phase comprising the cationic lipid, the phospholipid, the sterol and the PEG-lipid.

### BRIEF DESCRIPTION OF THE DRAWINGS

The accompanying drawings illustrate embodiments of the invention and, together with the description, serve to explain the invention. These drawings are offered by way of illustration and not by way of limitation.

FIG. 1 shows comparative analysis of day 1 (24 h post formulation) and day 10 (10^{th} day post formulation) activity of different lipid nanoparticle compositions encapsulating luciferase mRNA stored at 23±2° C.

### DETAILED DESCRIPTION

Unless defined otherwise, technical and scientific terms used herein have the same meaning as commonly understood by one of person skill in the art. Some of the terms are defined briefly here below; the definitions should not be construed in a limiting sense.

The singular forms “a”, “an” and “the” as used in the specification also include plural aspects unless the context dictates otherwise. Similarly, any singular term used in the specification also mean plural or vice versa unless the context dictates otherwise. As used herein in the claim(s), when used in conjunction with the word “comprising”, the words “a” or “an” may mean one or more than one. As used herein “another” may mean at least a second or more.

It must be noted that the words “comprising” or any of its form such as “comprise” or “comprises”, “having” or any of its forms such as “have” or “has”, “including” or any of its forms such as “include” or “includes”, or “containing” or any of its forms such as “contains” or “contains” are open-ended and do not exclude additional unrecited elements or method steps.

Wherever any quantity or range is stated one skilled in the art will recognize that quantity or range within 10 or 20 percent of the stated values can also be expected to be appropriate and reasonable and included within the scope of the invention.

Unless otherwise defined herein, scientific and technical terms used in connection with the present invention shall have the meanings that are commonly understood by those of ordinary skilled in the art. Generally, nomenclatures used in connection with, and techniques of, cell and tissue culture, molecular biology, immunology, microbiology, protein, adjuvant, pharmaceutical biotechnology, and biopharmaceutical manufacturing described herein are those well known and commonly used in the art. The methods and techniques of the present invention are generally performed according to conventional methods well known in the art and as described in various general and more specific references that are cited and discussed throughout the present specification.

The term “composition” or “formulation” has been used interchangeably to mean a lipid nanoparticle composition comprising a nucleic acid, an ionizable polymer, and lipid components such as cationic lipid, phospholipid, sterol, and PEG-lipid. The composition may additionally contains pharmaceutical carriers or excipients, such as but not limited to, buffering agents, stabilizers, tonicity modifiers, surfactants, chelating agents, salts, anti-oxidants, diluents, and/or preservatives or combinations thereof.

The term “derivative” as used herein means a compound that may be produced from another compound of similar structure in one or more steps. Derivative is generally formed from a similar beginning compound by attaching another molecule or atom to the beginning compound.

The term “therapeutic”, “therapeutic agent”, “prophylactic”, “prophylactic agent’, or drug has been used interchangeably to mean a compound or composition (such as a lipid nanoparticle composition described herein) having a biological effect or a combination of biological effects that prevents, inhibits, eliminates or prevents the progression of a disease or other aberrant biological processes in a subject, for example, an animal or human.

The term “preventing” is art-recognized, and when used in relation to a condition, such as an infection is well understood in the art, and includes administration of a composition, which reduces the frequency or severity, or delays the onset, of one or more symptoms of the medical condition in a subject relative to a subject who does not receive the composition. Thus, the prevention of a condition, such as an infection, includes, for example, the reduction of the frequency or severity of one or more symptoms of the medical condition in a population of patients receiving a therapy relative to a control population that did not receive the therapy, e.g., by a statistically and/or clinically significant amount. Similarly, the prevention of an infection includes reducing the likelihood that a patient receiving a therapy will develop the infection or related symptoms, relative to a patient who does not receive the therapy.

The term “stable” as used herein means a composition which retains an acceptable degree of physical stability, chemical stability and/or biological activity upon storage for a specified period of time at a given temperature. Stability of the therapeutic may be measured by techniques known to the person skilled in the art, for example, by SDS PAGE, dynamic light scattering, or immunogenicity assays. A composition may be stable even though the nucleic acid contained therein does not maintain 100% of its structure and/or function and/or biological activity after storage for a defined amount of time. Under certain circumstances, maintenance of at least about 80%, at least about 85%, at least about 90%, at least about 95%, at least about 96%, at least about 97%, at least about 98% or at least about 99% of nucleic acid's structure and/or function and/or biological activity after storage for a defined amount of time may be regarded as “stable.” In some embodiments, maintenance of about 30% to about 40%, about 40% to about 50%, about 50% to about 60%, about 60% to about 70%, or about 70% to about 80% of nucleic acid's structure and/or function and/or biological activity after storage for a defined amount of time may be regarded as “stable”.

The “specified period of time” as used herein means at least about 1 week, at least about 2 weeks, at least about 3 weeks, at least about 4 weeks, at least about 5 weeks, at least about 6, weeks, at least about 7 weeks, at least about 8 weeks, at least about 9 weeks, at least about 10 weeks, at least about 12 weeks, at least about 12 weeks, at least about 14 weeks, at least about 16 weeks, at least about 18 weeks, at least about 20 weeks, at least about 22 weeks, at least about 24 weeks, at least about 28 weeks, at least about 32 weeks, at least about 36 weeks, at least about 40 weeks, at least about 44 weeks, at least about 48 weeks, at least about 52 weeks, or more. In some embodiments, specified period of time also means at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 14 months, at least about 16 months, at least about 18 months, at least about 20 months, at least bout 22 months, at least about 24 months or more. In some embodiments, specified period of time also means at least about 5 days, at least about 6 days, at least about 7 days, at least about 8 days, at least about 10 days, at least about 11 days, at least about 12 days, at least about 13 days, at least about 14 days or more.

The term “molar ratio”, “mol ratio”, “molar percent”, “mol percent”, “molar %”, or “mol %” have been used interchangeably to mean number of moles of a component expressed as percentage relative to total moles of all lipid components (such as cationic lipid, phospholipid, sterol and PEG-lipid) and ionizable polymer component(s) present in the lipid nanoparticle compositions described herein. For example, 50 mol % of cationic lipid means, 50 mol % of cationic lipid is present in the lipid nanoparticle composition and other lipids components and ionizable polymer components together constitute the remaining 50 mol % such that the total amount of all the lipid components and ionizable polymer components constitute 100 mol %.

The terms “protein” or “peptide” and “polypeptide” have been used interchangeably herein and mean a polymer of amino acids linked through peptide bonds, but does not imply any specific length. The term also includes fusion proteins, muteins, analogs or modified forms.

The terms “antibody” and “antibodies” have been used interchangeably herein and means any antibody or antibody fragment (whether produced naturally or recombinantly) which retains antigen binding activity. This includes a monoclonal or polyclonal antibody, a single chain antibody, a Fab fragment of a monoclonal or polyclonal antibody, a chimeric antibody, a humanized antibody, a human antibody, a bispecific antibody, a multispecific antibody, or a nanobody.

The term “buffer” as used herein means those agents that maintains the pH of a solution in a desired range.

The term “cell” as used herein means a single cell or a population of cells or plurality of cells.

The term “biologically effective amount” or “therapeutically effective amount” as used herein means an amount of an agent, for example, a therapeutic, drug, therapeutic agent, prophylactic agent, diagnostic agent, composition, etc., that is sufficient, when administered to a subject suffering from or susceptible to an infection, disease, disorder, and/or condition, to treat, prevent, diagnose, improve symptoms of, and/or delay the onset of the infection, disease, disorder, and/or condition. A therapeutically effective amount herein may vary according to factors such as the disease state, age, sex, and weight of the patient.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment. Treatment may be administered to a subject who does not exhibit signs of a disease and/or exhibits only early signs of the disease for the purpose of decreasing the risk of developing pathology associated with the disease.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

The term “disease” as used herein, means an interruption, cessation, or disorder of body function, system, or organ. Non limiting examples of disease include malignant diseases, autoimmune diseases, inherited diseases, metabolic disorders, or infectious diseases.

As used herein, administration “conjointly” with another compound or composition includes simultaneous administration and/or administration at different times. Conjoint administration also encompasses administration as a co-formulation or administration as separate compositions, including at different dosing frequencies or intervals, and using the same route of administration or different routes of administration.

### Lipid Nanoparticle (LNP) Composition

Lipid nanoparticle compositions described herein typically comprise a nucleic acid, an ionizable polymer, a cationic lipid, a phospholipid, a sterol, and a PEG-lipid.

In some aspects, a lipid nanoparticle composition comprising a nucleic acid, a biocompatible polymer, a cationic lipid, a phospholipid, a sterol and a PEG-lipid is disclosed herein.

In some aspects, a lipid nanoparticle composition comprising a nucleic acid, an ionizable polymer in an amount from 1 mol percent to 25 mol percent, an ionizable lipid, a phospholipid, a sterol and a PEG-lipid is disclosed herein.

In some aspects, a lipid nanoparticle composition comprising a nucleic acid, an ionizable polymer in an amount from 1 mol percent to 25 mol percent, an ionizable lipid in an amount from 25 mol percent to 50 mol percent, a phospholipid, a sterol and a PEG-lipid is disclosed herein.

In some aspects, a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 2 mol percent to 20 mol percent, a sterol and a PEG-lipid is disclosed herein.

In some embodiments, the disclosure relates to a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid.

In some embodiments, the disclosure relates to a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 1 mol percent to 20 mol percent, a sterol in an amount from 30 mol percent to 65 mol percent and a PEG-lipid in an amount from 0.2 mol percent to 2 mol percent.

In some embodiments, the disclosure relates to a lipid nanoparticle composition comprising a nucleic acid, an ionizable polymer selected from chitosan, chitosan derivatives, cellulose derivatives, poly-L-lysine, poly-L-glutamic acid, or combination thereof in an amount from 1 mol percent to 25 mol percent, an ionizable lipid, a phospholipid, a sterol and a PEG-lipid.

In some embodiments, the disclosure relates to a lipid nanoparticle composition comprising a nucleic acid, an ionizable polymer selected from chitosan, chitosan derivatives, cellulose derivatives, poly-L-glutamic acid, or combination thereof in an amount from 1 mol percent to 25 mol percent, an ionizable lipid, a phospholipid, a sterol and a PEG-lipid.

In some embodiments, the disclosure relates to a lipid nanoparticle composition comprising a nucleic acid, an ionizable polymer selected from chitosan, chitosan derivatives, cellulose derivatives or combination thereof in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid in an amount from 0.2 mol percent to 2 mol percent.

In some embodiments, the disclosure relates to a lipid nanoparticle composition comprising a nucleic acid, an ionizable polymer selected from chitosan and/or its derivatives or combination thereof in an amount from 1 mol percent to 25 mol percent, an ionizable lipid, a phospholipid, a sterol and a PEG-lipid.

In some embodiments, the disclosure relates to a lipid nanoparticle composition comprising a nucleic acid, a chitosan or chitosan derivative or combination thereof in an amount from 5 mol percent to 15 mol percent, a cationic lipid, a phospholipid, a sterol and a PEG-lipid.

In some embodiments, the disclosure relates to a pharmaceutical composition which includes a lipid nanoparticle composition described herein and a pharmaceutically acceptable carrier or excipients.

In some embodiments, the disclosure relates to a method of delivering a nucleic acid comprising administering the lipid nanoparticle composition comprising a nucleic acid, an ionizable polymer, a cationic lipid, a phospholipid, a sterol and a PEG-lipid of the present disclosure.

In some embodiments, the disclosure relates to a method of treating or preventing a disease, comprising administering to a subject in need thereof a lipid nanoparticle composition comprising a nucleic acid, an ionizable polymer, a cationic lipid, a phospholipid, a sterol, and a PEG-lipid.

In some embodiments, the disclosure relates to a method of treating or preventing a disease, comprising administering to a subject in need thereof a lipid nanoparticle composition comprising a nucleic acid, an ionizable polymer, an ionizable lipid, a phospholipid, a sterol, and a PEG-lipid.

In some embodiments, the disclosure relates to a method of treating or preventing a disease, comprising administering to a subject in need thereof a lipid nanoparticle composition comprising a nucleic acid, an ionizable polymer in an amount from 1 mol percent to 25 mol percent, an ionizable lipid, a phospholipid, a sterol, and a PEG-lipid.

In some embodiments, the disclosure relates to a method of treating or preventing a disease, comprising administering to a subject in need thereof a lipid nanoparticle composition comprising a nucleic acid, an ionizable polymer in an amount from 1 mol percent to 25 mol percent, an ionizable lipid in an amount from 25 mol percent to 50 mol percent, a phospholipid, a sterol, and a PEG-lipid.

In some embodiments, the disclosure relates to a method of treating or preventing a disease, comprising administering to a subject in need thereof a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 2 mol percent to 20 mol percent, a sterol and a PEG-lipid.

In some embodiments, the disclosure relates to a method of treating or preventing a disease, comprising administering to a subject in need thereof a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid.

In some embodiments, the disclosure relates to a method of treating or preventing a disease, comprising administering to a subject in need thereof a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid in an amount from 0.2 mol percent to 2 mol percent.

In some embodiments, the disclosure relates to a method of treating or preventing a disease, comprising administering to a subject in need thereof a lipid nanoparticle composition comprising a nucleic acid, an ionizable polymer selected from chitosan, chitosan derivatives, cellulose derivatives, poly-L-glutamic acid, or combination thereof in an amount from 1 mol percent to 25 mol percent, an ionizable lipid, a phospholipid, a sterol and a PEG-lipid.

In some embodiments, the disclosure relates to a method of treating or preventing a disease, comprising administering to a subject in need thereof a lipid nanoparticle composition comprising a nucleic acid, an ionizable polymer selected from chitosan, chitosan derivatives, cellulose derivatives or combination thereof in an amount from 1 mol percent to 25 mol percent, an ionizable lipid, a phospholipid, a sterol and a PEG-lipid.

In some embodiments, the disclosure relates to a method of treating or preventing a disease, comprising administering to a subject in need thereof a lipid nanoparticle composition comprising a nucleic acid, an ionizable polymer selected from chitosan, chitosan derivatives, cellulose derivatives or combination thereof in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid in an amount from 0.2 mol percent to 2 mol percent.

In some embodiments, the disclosure relates to a method of treating or preventing a disease, comprising administering to a subject in need thereof a lipid nanoparticle composition comprising a nucleic acid, an ionizable polymer selected from chitosan and/or its derivatives or combination thereof in an amount from 1 mol percent to 25 mol percent, an ionizable lipid, a phospholipid, a sterol and a PEG-lipid.

In some embodiments, the disclosure relates to use of a lipid nanoparticle composition comprising a nucleic acid, an ionizable polymer, a cationic lipid, a phospholipid, a sterol, and a PEG-lipid in the manufacture of a medicament for the treatment or prevention of a disease in a subject.

In some embodiments, the disclosure relates to use of a lipid nanoparticle composition comprising a nucleic acid, an ionizable polymer, an ionizable lipid, a phospholipid, a sterol, and a PEG-lipid in the manufacture of a medicament for the treatment or prevention of a disease in a subject.

In some embodiments, the disclosure relates to use of a lipid nanoparticle composition comprising a nucleic acid, an ionizable polymer in an amount from 1 mol percent to 25 mol percent, an ionizable lipid, a phospholipid, a sterol, and a PEG-lipid in the manufacture of a medicament for the treatment or prevention of a disease in a subject.

In some embodiments, the disclosure relates to use of a lipid nanoparticle composition comprising a nucleic acid, an ionizable polymer in an amount from 1 mol percent to 25 mol percent, an ionizable lipid in an amount from 25 mol percent to 50 mol percent, a phospholipid, a sterol, and a PEG-lipid in the manufacture of a medicament for the treatment or prevention of a disease in a subject.

In some embodiments, the disclosure relates to use of a lipid nanoparticle composition comprising a nucleic acid, an ionizable polymer in an amount from 1 molar percent to 25 mol percent, an ionizable lipid in an amount from 25 mol percent to 50 mol percent, a phospholipid in an amount from 2 mol percent to 20 mol percent, a sterol and a PEG-lipid in the manufacture of a medicament for the treatment or prevention of a disease in a subject.

In some embodiments, the disclosure relates to use of a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid in the manufacture of a medicament for the treatment or prevention of a disease in a subject.

In some embodiments, the disclosure relates to use of a lipid nanoparticle composition comprising a nucleic acid, an ionizable polymer in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid in an amount from 0.2 mol percent to 2 mol percent in the manufacture of a medicament for the treatment or prevention of a disease in a subject.

In some embodiments, the disclosure relates to use of a lipid nanoparticle composition comprising a nucleic acid, an ionizable polymer selected from chitosan, chitosan derivatives, cellulose derivatives, poly-L-lysine, poly-L-glutamic acid, or combination thereof in an amount from 1 mol percent to 25 mol percent, an ionizable lipid, a phospholipid, a sterol and a PEG-lipid in the manufacture of a medicament for the treatment or prevention of a disease in a subject.

In some embodiments, the disclosure relates to use of a lipid nanoparticle composition comprising a nucleic acid, an ionizable polymer selected from chitosan, chitosan derivatives, cellulose derivatives or combination thereof in an amount from 1 mol percent to 25 mol percent, an ionizable lipid, a phospholipid, a sterol and a PEG-lipid in the manufacture of a medicament for the treatment or prevention of a disease in a subject.

In some embodiments, the disclosure relates to use of a lipid nanoparticle composition comprising a nucleic acid, an ionizable polymer selected from chitosan, chitosan derivatives, cellulose derivatives or combination thereof in an amount from 1 mol percent to 25 mol percent, an ionizable lipid in an amount from 25 mol percent to 50 mol percent, a phospholipid in an amount from 2 mol percent to 20 mol percent, a sterol in an amount from 30 mol percent to 65 mol percent and a PEG-lipid in an amount from 0.2 mol percent to 2 mol percent in the manufacture of a medicament for the treatment or prevention of a disease in a subject.

In some embodiments, the disclosure relates to use of a lipid nanoparticle composition comprising a nucleic acid, an ionizable polymer selected from chitosan and/or its derivatives or combination thereof in an amount from 1 mol percent to 25 mol percent, an ionizable lipid, a phospholipid, a sterol and a PEG-lipid in the manufacture of a medicament for the treatment or prevention of a disease in a subject.

In some embodiments, the disclosure relates to a method of delivering nucleic acid to a cell, comprising delivering a lipid nanoparticle composition to the cell, wherein the lipid nanoparticle composition comprises a nucleic acid, an ionizable polymer, a cationic lipid, a phospholipid, a sterol and a PEG-lipid.

In some embodiments, the disclosure relates to a method of preparing a lipid nanoparticle composition, comprising mixing an aqueous phase comprising a nucleic acid and an ionizable polymer, and an organic phase comprising an cationic lipid, a phospholipid, a sterol and a PEG-lipid.

### Nucleic Acid

The term “nucleic acid” as used herein means a polymer comprising two or more nucleotides for example, deoxyribonucleotides or ribonucleotides, either in an unmodified or modified form. The nucleic acid may be either single stranded or double stranded, linear or circular.

The term “nucleotide” as used herein means a ribonucleotide or deoxyribonucleotide. If the term nucleotide is used in the context of RNA, it refers to ribonucleotide, and if it is used in the context of DNA, it refers to deoxyribonucleotide.

The term “ribonucleic acid” or “RNA” has been used interchangeably herein and means a polymer of ribonucleotides. The RNA may be either single stranded or double stranded, linear or circular. The term RNA also includes messenger RNA (mRNA), and non-coding RNA (ncRNA).

The term “deoxyribonucleic acid” or “DNA” has been used interchangeably herein and means a polymer of deoxyribonucleotides. The DNA may be either single stranded or double stranded, linear or circular.

In some embodiments, the nucleic acid encodes an antigen such as, but not limited to: those derived from Cholera toxoid, tetanus toxoid, diphtheria toxoid, hepatitis B surface antigen, hemagglutinin, neuraminidase, influenza M protein, PfHRP2, pLDH, aldolase, MSP1, MSP2, AMA1, Der-p-1, Der-f-1, Adipophilin, AFP, AIM-2, ART-4, BAGE, alpha-fetoprotein, BCL-2, Bcr-Abl, BING-4, CEA, CPSF, CT, cyclin DIEp-CAM, EphA2, EphA3, ELF-2, FGF-5, G250, Gonadotropin Releasing Hormone, HER-2, intestinal carboxyl esterase (iCE), IL13Ralpha2, MAGE-1, MAGE-2, MAGE-3, MART-1, MART-2, M-CSF, MDM-2, MMP-2, MUC-1, NY-EOS-1, MUM-1, MUM-2, MUM-3, p53, PBF, PRAME, PSA, PSMA, RAGE-1, RNF43, RU1, RU2AS, SART-1, SART-2, SART-3, SAGE-1, SCRN 1, SOX2, SOXIO, STEAP1, survivin (BIRC5), Telomerase, TGFbetaR11, TRAG-3, TRP-1, TRP-2, TERT, or WT1; those derived from a virus, such as Cowpoxvirus, Vaccinia virus, Pseudocowpox virus, Human herpesvirus 1, Human herpesvirus 2, Cytomegalovirus, Human adenovirus A-F, Polyomavirus, Human papillomavirus, Parvovirus, Hepatitis A virus, Hepatitis B virus, Hepatitis C virus, Human immunodeficiency virus, Orthoreo virus, Rotavirus, Ebolavirus, parainfluenza virus, influenza virus (e.g., H5N1 influenza virus, influenza A virus, influenza B virus, influenza C virus), Measles virus, Mumps virus, Rubella virus, Pneumovirus, Human respiratory syncytial virus, Rabies virus, California encephalitis virus, Japanese encephalitis virus, Hantaan virus, Lymphocytic choriomeningitis virus, Coronavirus (e.g., SARS-CoV-2), Enterovirus, Rhino virus, Poliovirus, Norovirus, Flavivirus, Dengue virus, West Nile virus, Yellow fever virus and varicella; those derived from a bacterium, such as Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli 0151: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica,’ or those derived from a protozoa, e.g., of the genus Plasmodium (Plasmodium falciparum, Plasmodium malariae, Plasmodium vivax, Plasmodium ovale or Plasmodium knowlesi.

The antigen may be an allergen derived from, without limitation, cells, cell extracts, proteins, polypeptides, peptides, peptide mimics of polysaccharides and other molecules, such as small molecules, lipids, glycolipids, and carbohydrates of plants, animals, fungi, insects, food, drugs, dust, and mites. Allergens include but are not limited to environmental aeroallergens; plant pollens (e.g., ragweed/hayfever); weed pollen allergens; grass pollen allergens; Johnson grass; tree pollen allergens; ryegrass; arachnid allergens (e.g., house dust mite allergens); storage mite allergens; Japanese cedar pollen/hay fever; mold/fungal spore allergens; animal allergens (e.g., dog, guinea pig, hamster, gerbil, rat, mouse, etc., allergens); food allergens (e.g., crustaceans; nuts; citrus fruits; flour; coffee); insect allergens (e.g., fleas, cockroach); venoms: (Hymenoptera, yellow jacket, honey bee, wasp, hornet, fire ant); bacterial allergens (e.g., streptococcal antigens; parasite allergens such as Ascaris antigen); viral antigens; drug allergens; hormones (e.g., insulin); enzymes (e.g., streptokinase); and drugs or chemicals capable of acting as incomplete antigens or haptens (e.g., the acid anhydrides and the isocyanates). Where a hapten is used in a composition of the disclosure, it may be attached to a carrier to form a hapten-carrier adduct. The hapten-carrier adduct is capable of initiating a humoral immune response, whereas the hapten itself would not elicit antibody production. Non-limiting examples of haptens are aniline, urushiol (a toxin in poison ivy), hydralazine, fluorescein, biotin, digoxigenin and dinitrophenol.

In other embodiments, the antigen is an antigen associated with a disease where it is desirable to sequester the antigen in circulation, such as for example an amyloid protein (e.g., Alzheimer's disease).

In some embodiments, the nucleic acid regulates or modulates cellular functions.

The term “cellular functions” as used herein means various cellular or biological processes such as, but not limited to, biosynthesis, cell division, cell cycle regulation, cellular metabolism, ion transport, absorption, secretion, homeostasis, replication, transcription, translation, cell signalling, endocytosis, exocytosis, phagocytosis, apoptosis, DNA replication, DNA repair, protein synthesis, gene regulation, cell repair, cell growth, cell differentiation, cellular trafficking, cell proliferation, metabolic pathways etc.

The terms “regulate” or “modulate” or “regulation” or “modulation” have been used interchangeably herein and means an act of controlling a cellular or biological process or to exert a modifying or controlling influence on cellular or biological process.

Messenger RNA (mRNA)

Messenger RNA (mRNA) is a polymer of ribonucleotides that encodes at least a protein or polypeptide or peptide. Typically, an mRNA includes at least a coding region, a 5′ UTR, a 3′ UTR, a 5′ cap and a poly(A) tail. UTR (untranslated regions) flanks the coding region or open reading frame (ORF). The 5′ UTR and the 3′ UTR are sections of the mRNA before the start codon and after the stop codon respectively. The 5′ UTR has a cap (5′ cap) consisting of altered nucleotides. mRNA also contains a polyadenylated region at its 3′ end having adenine nucleotides called poly(A) tail.

In some embodiments, the mRNA may be unmodified or modified or combination of both. The modification may be in the nucleobase of the nucleotide, or sugar moiety of the nucleotide, or the phosphate of the nucleotide. In some embodiments, unmodified mRNA may comprise naturally occurring nucleosides, for example, adenosine, guanosine, cytidine, and uridine. mRNA may comprise one or more modified nucleosides, for example, adenosine analog, guanosine analog, cytidine analog, or uridine analog.

In some embodiments, the one or more modified nucleosides is a nucleoside analog selected from 2-aminoadenosine, 3-methyl adenosine, 7-deazaadenosine, 7-deazaguanosine, 8-oxoadenosine, or 8-oxoguanosine.

In some embodiments, the one or more modified nucleosides is a uridine analog selected from propynyl-uridine, pseudouridine, C5-bromouridine, C5-fluorouridine, C5-iodouridine, C5-propynyl-uridine, 5-aza-uridine, 2-thio-5-aza-uridine, 2-thio-uridine, 4-thio-pseudouridine, 2-thio-pseudouridine, 5-hydroxy-uridine, 3-methyl-uridine, 5-carboxymethyl-uridine, 1-carboxymethyl-pseudouridine, 1-methyl-3-(3-amino-3-carboxypropyl)pseudouridine, 2-thio-2′-O-methyl-uridine, 5-methoxycarbonylmethyl-2′-O-methyl-uridine, 5-carboxymethylaminomethyl-2′-O-methyl-uridine, 3,2′-O-dimethyl-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurino-4-thio-pseudouridine, 1-methyl-pseudouridine, 4-thio-1-methyl-pseudouridine, 2-thio-1-methyl-pseudouridine, 1-methyl-1deaza-pseudouridine, 2-thio-1-methyl-1-deaza-pseudouridine, dihydro-uridine, dihydro-pseudouridine, 2-thio-dihydro-uridine, 2-thio-dihydro-pseudouridine, 2-methoxy-uridine, 2-methoxy-4-thio-uridine, 4-methoxy-pseudouridine, or 4-methoxy-2-thio-pseudouridine.

In some embodiments, the one or more modified nucleosides is a cytidine analog selected from 5-methylcytidine, C5-propynyl-cytidine, C5-methylcytidine, pseudoisocytidine, 1-methyl-pseudoisocytidine, pyrrolo-pseudoisocytidine, 4-thio-pseudoisocytidine, 4-thio-1-methyl-pseudoisocytidine, 4-thio-1-methyl-1-deaza-pseudoisocytidine, 1-methyl-1-1deaza-pseudoisocytidine, 4-methoxy-1-methyl-pseudoisocytidine or combinations thereof.

Methods for making modified nucleosides are well known in the art (WO 2020168466, U.S. Pat. Nos. 8,278,036; 8,691,966; 8,748,089; 8,835,108; 9,750,824; U.S. Ser. No. 10/232,055; WO2007024708; WO2012135805; WO2013052523; WO2011012316) In some embodiments, the modified nucleoside is pseudouridine, for example, 1-methyl-pseudouridine, 1-propynyl-pseudouridine, 1-carboxymethyl-pseudouridine, 1-methyl-3-(3-amino-3-carboxypropyl)pseudouridine, 4-methoxy-pseudouridine, or 4-methoxy-2-thio-pseudouridine 4-thio-pseudouridine, 2-thio-pseudouridine, 4-thio-1-methyl-pseudouridine, 2-thio-1-methyl-pseudouridine, dihydro-pseudouridine or combination thereof.

In some embodiments, mRNA is obtained from natural sources (i.e., isolated from the cells), produced using recombinant expression system, or chemically synthesized. mRNAs according to the present disclosure may be synthesized via in vitro transcription (IVT). Briefly, IVT is typically performed with a DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions may vary according to the specific application. Methods of making mRNA through IVT reaction is well known in the art (see for example, Beckert, Bertrand and Masquida, Benoit Methods in Molecular Biology (2011) 703, 29-41; Brunelle, Julie L. and Green Rachel Methods in Enzymology (2013) 530, 101-114; Kamakaka, Rohinton T. and Kraus W. Lee Current Protocols in Cell Biology (1999) 11.6.1-11.6.17; Kanwal, Fariha et al. Cellular Physiology and Biochemistry (2018) 48:1915-1927; WO2018157153; WO2020185811; WO2022082001)

In some embodiments, the in vitro transcription occurs in a single batch. In some embodiments, IVT reaction includes capping and tailing reactions either co-transcriptionally or separately. A cap analog is added to the in vitro transcription reaction and will be incorporated at the 5′ end of the mRNA during the reaction. Alternative method of capping involves adding the cap post-transcriptionally through an enzymatic reaction. The poly (A) tail can be incorporated into the DNA template sequence, and thus the poly (A) tail will be incorporated into the mRNA by T7 RNA polymerase during the in vitro transcription. Alternative method of tailing involves adding the poly (A) tail post-transcriptionally through an enzymatic reaction. In some embodiments, capping and tailing reactions are performed co-transcriptionally i.e., during the IVT reaction. In some embodiments, capping and tailing reactions are performed separately from IVT reaction. mRNA produced as a result of IVT reaction may be purified using techniques well known in the art, such as, centrifugation, filtration and/or chromatographic techniques. The purification of mRNA may be accomplished before capping and tailing steps are performed or after capping and tailing. The synthesized mRNA may be purified by ethanol precipitation or filtration or chromatography methods. In some embodiments, tangential flow filtration is used to purify mRNA. In some embodiments, mRNA is purified by chromatographic step. In other embodiments, mRNA is purified by a combination of filtration and chromatography steps.

In some embodiments, a suitable mRNA sequence is an mRNA sequence encoding a protein, peptide, polypeptide or an antibody. In some embodiments, a suitable mRNA sequence is codon optimized for efficient expression in a host cell or organism. Codon optimization typically includes modifying a naturally-occurring or wild-type nucleic acid sequence encoding a peptide, polypeptide or protein to achieve the highest possible expression of peptide, polypeptide, protein or an antibody without altering the amino acid sequence.

Any length of mRNA can be encapsulated in the lipid nanoparticles of the present disclosure. The length of the mRNA used in the lipid nanoparticle of the present disclosure depends on the gene product or protein or protein fragment to be incorporated in the lipid nanoparticle. Thus, mRNA can be very short extending to about a few hundred nucleotides in length or very long extending to about several thousand nucleotides in length. In some embodiments, mRNA is about 0.5 kb, 1 kb, 1.5 kb, 2 kb, 2.5 kb, 3 kb, 3.5 kb, 4 kb, 4.5 kb, 5.5 kb 6 kb, 6.5 kb, 7 kb, 7.5 kb, 8 kb, 8.5 kb, 9 kb, 9.5 kb, 10 kb, 11 kb, 12 kb, 13, kb, 14, kb, 15 kb, 16 kb, 17 kb, 18 kb, 19 kb, 20 kb, 22 kb, 24, kb, 26 kb, 28 kb, or 30 kb in length. In other embodiments, mRNA is about 0.5 to 30 kb, 0.5 to 25 kb, 0.5 to 20 kb in length. In still other embodiments, mRNA is about 1 to 20 kb, 1 to 15 kb, or 1 to 10 kb in length.

In some embodiments, the mRNA is circular. In other embodiments, the mRNA is linear.

In some embodiments, the mRNA is self-amplifying or self-replicating. Self-amplifying or self-replicating mRNA as used herein means an mRNA that self-replicate upon delivery into the cells. Such mRNAs typically contain a replicase, usually derived from an alphavirus, which enables amplification of the original strand of mRNA encoding the protein of interest upon delivery into the cells (Beissert, Tim et al. Molecular Therapy 2020 28:119-128). mRNA present in the lipid nanoparticle composition may be present in a biologically effective amount or therapeutically effective amount. In some embodiments, the biologically effective amount of mRNA present in the lipid nanoparticle composition is between 0.1 μg to 1000 μg, 0.1 μg to 950 μg, 0.1 μg to 900 μg, 0.1 μg to 850 μg, 0.1 μg to 800 μg, 0.1 μg to 750 μg, 0.1 μg to 700 μg, 0.1 μg to 650, 0.1 μg to 600, 0.1 g to 550, 0.1 g to 500 μg, 0.1 to 450 μg, 0.1 μg to 400 μg, 0.1 μg to 350 μg, 0.1 to 300 μg, 0.1 to 200 μg or any range therein. In some embodiments, the biologically effective amount of mRNA present in the lipid nanoparticle composition is from about 0.1 μg to 1000 μg, 0.1 μg to 950 μg, 0.1 μg to 900 μg, 0.1 μg to 850 μg, 0.1 μg to 800 μg, 0.1 μg to 750 μg, 0.1 μg to 700 μg, 0.1 μg to 650, 0.1 μg to 600, 0.1 μg to 550, 0.1 μg to 500 μg or any range therein.

In some embodiments, the biologically effective amount of mRNA present in the lipid nanoparticle composition is 0.1 μg, 0.2 μg, 0.3 μg, 0.4 μg, 0.5 μg, 0.6 μg, 0.7, μg, 0.8 μg, 0.9 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 100 μg, 110 μg, 120 μg, 130 μg, 140 μg, 150 μg, 160 μg, 170 μg, 180 μg, 190 μg, 200 μg, 220 μg, 240 μg, 250 μg, 260 μg, 280 μg, 300 μg, 350 μg, 400 μg, 450 μg, 500 μg, 600 μg, 700 μg, 800 μg, 900 μg, 1000 μg or any portion or fraction thereof.

In some embodiments, the mRNA encodes one or more proteins, one or more antibodies, or combination thereof. The mRNA encoding one or more proteins, or one or more antibodies may belong to any organism such as a prokaryote or a eukaryote, a unicellular organism, a multicellular organism, a virus, a bacterium, a mycoplasma, a protozoan, an animal or a human.

Non-Coding RNA (ncRNA)

The term “non-coding RNA” or “ncRNA” has been used interchangeably to mean any RNA molecule that is not generally translated, but sometimes can, into a polypeptide or protein and includes, long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA) and ribosomal RNA (rRNA). The term ncRNA also includes such RNAs that encode small peptides such as lncRNA.

In some embodiments, the nucleic acid component of the lipid nanoparticle compositions comprises ncRNA. The ncRNA may be naturally occurring or wild type. In other embodiments, the ncRNA may be synthetically produced.

In some embodiments, the ncRNA is single stranded or double stranded.

In some embodiments, the ncRNA is a few nucleotides long to several thousand nucleotides long.

In some embodiments, the ncRNA comprises long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA), ribosomal RNA (rRNA) or combination thereof.

In some embodiments, the long non-coding RNA (lncRNA) is more than 200 nucleotide long.

### Deoxyribonucleic Acid (DNA)

Any DNA molecule capable of transferring a gene into a cell, for example to express a transcript, can be incorporated into the lipid nanoparticle compositions described herein.

The term “DNA sequence” or “DNA segment” or “gene” has been used interchangeably and mean a segment or sequence of DNA capable of being used to produce a transcript which may either be a messenger RNA (mRNA) or non-coding RNAs (ncRNAs) such as long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA) or ribosomal RNA (rRNA).

In some embodiments, the DNA molecule is obtained form natural sources. In other embodiments, the DNA molecule is recombinantly or synthetically produced.

In some embodiments, the DNA molecule is modified or unmodified, linear or circular.

In some embodiments, the DNA molecule is double stranded or single stranded.

In some embodiments, the DNA molecule includes a coding sequence or a non-coding sequence.

In some embodiments, the DNA molecule is a few nucleotides long to several thousand nucleotides long.

### Ionizable Polymer

As used herein the term “polymer” means a compound formed from a plurality of repeating units called monomers. Polymers are produced through a process called polymerization wherein two or more monomers are linked through chemical bonds to form the polymer. In some embodiments, the polymer is branched or unbranched. In some embodiments, the polymer may be homopolymer, i.e., consisting of same type of repeat units or monomers, or heteropolymer, i.e., consisting of more than one type of repeat units or monomers. The terms heteropolymer and copolymer have been used interchangeably herein.

The term “ionizable polymer” as used herein means, a polymer that can exist in a positively charged or neutral form depending on the pH of the solution or environment, for example, ionizable polymer will be cationic (positively charged) around acidic pH (pH 1.0 to pH 6.9) and neutral (no charge) around physiological pH (pH 7.0 to pH 7.5).

In some embodiments, the ionizable polymer is a biocompatible polymer or biodegradable polymer. The term “biocompatible polymer” and “biodegradable polymer” have been used interchangeably to mean a polymer that is substantially free from any deleterious effects when introduced into a living or biological system. Such polymers are capable of undergoing degradation when introduced into the living or biological systems and are not expected to produce significant toxicity or immunological response.

In some embodiments, the lipid nanoparticle compositions comprise an ionizable polymer. The ionizable polymer may be selected from chitosan, chitosan derivatives, cellulose derivatives, poly-L-lysine (PLL), poly-L-glutamic acid, protamine, polyethyleneimine, their derivatives, or combinations thereof.

In some embodiments, the ionizable polymer is positively charged at acidic pH i.e., pH 1.0 to 6.9 and is neutral around physiological pH (pH 7.0 to 7.5).

The proportion of ionizable polymer present in the lipid nanoparticle compositions may be from about 1 mol % to about 25 mol %.

In some embodiments, the proportion of ionizable polymer present in the lipid nanoparticle compositions is from about 1 mol % to about 25 mol %, 1 mol % to about 25 mol %, from about 1 mol % to about 24 mol %, from about 1 mol % to about 23 mol %, from about 1 mol % to about 22 mol %, from about 1 mol % to about 21 mol %, from about 1 mol % to about 20 mol %, from about 1 mol % to about 19 mol %, or from about 1 mol % to about 18 mol %, from about 1 mol % to about 17 mol %, from about 1 mol % to about 16 mol %, from about 1 mol % to about 15 mol % or any range therein.

In some embodiment, the proportion of ionizable polymer present in the lipid nanoparticle compositions is from about 1 mol % to about 25 mol %, from about 2 mol % to about 25 mol %, from about 3 mol % to about 25 mol %, from about 4 mol % to about 25 mol %, from about 5 mol % to about 25 mol %, from about 5 mol % to about 24 mol %, from about 5 mol % to about 23 mol %, from about 5 mol % to about 22 mol %, from about 5 mol % to about 21 mol %, from about 5 mol % to about 20 mol %, from about 5 mol % to about 19 mol %, or from about 5 mol % to about 18 mol %, from about 5 mol % to about 17 mol %, from about 5 mol % to about 16 mol %, from about 5 mol % to about 15 mol % or any range therein.

In some embodiments, the proportion of ionizable polymer present in the lipid nanoparticle compositions is about 1 mol %, is about 2 mol %, about 3 mol %, about 4 mol %, 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, or any portion or fraction thereof.

In some embodiments, the preferred ionizable polymer comprises chitosan, chitosan derivatives, cellulose derivatives, or combinations thereof.

### Chitosan

Chitosan is a natural polymer composed of glucosamine units. Chitosan is chemically poly-β-(1-4)-2-amino-2-deoxy-D-glucose. Chitosan is prepared by partial deacetylation of chitin, which is commonly carried out by alkaline hydrolysis. Thus, chitosan may contain acetylated units (N-acetyl-D-glucosamine) as well as deacetylated units (β-(1→4)-linked D-glucosamine). Typically, chitosan molecule has greater than 60% degree of deacetylation when compared to chitin. The molecular weight of chitosan typically varies between 10 kDa to 1000 kDa. Chitosan nanoparticles have been used for drug delivery, including delivery of nucleic acids. However, chitosan nanoparticles alone are insufficient in effective delivery of nucleic acids (Ragelle, Héloïse et al. Journal of Controlled Release (2013) 172: 207-218).

In some embodiments, the lipid nanoparticle composition comprises an ionizable polymer such as chitosan along with lipid components and nucleic acid.

In some embodiments, the ionizable polymer is chitosan or its derivatives. In some embodiments, the ionizable polymer includes chitosan derivatives or dialdehyde chitosan derivatives or combination thereof.

The chitosan or chitosan derivatives employed in the present disclosure may have a molecular weight from about 25 kDa to about 400 kDa.

In some embodiments, the molecular weight of chitosan or its derivatives is from about 25 kDa to 375 Kda, from about 30 kDa to about 350 kDa, from about from about 35 kDa to about 325 kDa, from about 40 kDa to about 300 kDa, from about 40 kDa to about 250 kDa, from about 40 kDa to about 225 kDa, from about 40 kDa to about 220 kDa, from about 40 kDa to about 210 kDa, or from about 40 kDa to about 200 kDa or any range therein.

The proportion of chitosan or its derivatives or combination thereof present in the lipid nanoparticle compositions may be from about 1 mol % to about 25 mol %.

In some embodiments, the proportion of chitosan or its derivatives or combination thereof present in the lipid nanoparticle compositions is from about 1 mol % to about 25 mol %, 1 mol % to about 25 mol %, from about 1 mol % to about 24 mol %, from about 1 mol % to about 23 mol %, from about 1 mol % to about 22 mol %, from about 1 mol % to about 21 mol %, from about 1 mol % to about 20 mol %, from about 1 mol % to about 19 mol %, or from about 1 mol % to about 18 mol %, from about 1 mol % to about 17 mol %, from about 1 mol % to about 16 mol %, from about 1 mol % to about 15 mol % or any range therein.

In some embodiments, the proportion of chitosan or its derivatives or combination thereof present in the lipid nanoparticle compositions is from about 1 mol % to about 25 mol %, from about 2 mol % to about 25 mol %, from about 3 mol % to about 25 mol %, from about 4 mol % to about 25 mol %, from about 5 mol % to about 25 mol %, from about 2 mol % to about 24 mol %, from about 2 mol % to about 23 mol %, from about 2 mol % to about 22 mol %, from about 2 mol % to about 21 mol %, from about 2 mol % to about 20 mol %, from about 2 mol % to about 19 mol %, or from about 2 mol % to about 18 mol %, from about 2 mol % to about 17 mol %, from about 2 mol % to about 16 mol %, from about 2 mol % to about 15 mol % or any range therein.

In some embodiments, the proportion of chitosan or its derivatives or combination thereof present in the lipid nanoparticle compositions is from about 1 mol % to about 25 mol %, preferably about 2 mol % to 20 mol %, most preferably about 5 mol % to about 15 mol %.

In some embodiments, the proportion of chitosan or its derivatives or combination thereof present in the lipid nanoparticle compositions is about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, or any portion or fraction thereof.

### Cellulose

Cellulose is a polymer composed of a linear chain of d-glucose units linked via β-1,4 glycosidic bonds. It typically contains repeating glucose units ranging from few hundreds to several thousands. Native cellulose is not ideal for mRNA delivery and therefore, requires modification in accordance with the present disclosure. In some embodiments, cellulose is modified (Jelkmann et al. Biomacromolecules (2018) 19: 4059-4067; Lee, Hye Ji et al. International Journal of Biosciences Biochemistry and Bioinformatics (2019) 9: 134-140). Cellulose and cellulose derived materials have been used for drug delivery of small molecules (Amalin Kavitha, K. Thomas Paul, Parambath Anilkumar, Chapter 18—Cellulose-derived materials for drug delivery applications, Editor(s): Faruq Mohammad, Hamad A. Al-Lohedan, Mohammad Jawaid, In Micro and Nano Technologies, Sustainable Nanocellulose and Nanohydrogels from Natural Sources, Elsevier, 2020, Pages 367-390, ISBN 9780128167892).

In some embodiments, the lipid nanoparticle composition comprises cellulose derivatives. Cellulose derivatives may be dialdehyde cellulose derivatives.

The proportion of cellulose derivatives or dialdehyde cellulose derivatives or combination thereof present in the lipid nanoparticle compositions may be from about 1 mol % to about 25 mol %.

In some embodiments, the proportion of cellulose derivatives or dialdehyde cellulose derivatives or combination thereof present in the lipid nanoparticle compositions is from about 1 mol % to about 25 mol %, 1 mol % to about 25 mol %, from about 1 mol % to about 24 mol %, from about 1 mol % to about 23 mol %, from about 1 mol % to about 22 mol %, from about 1 mol % to about 21 mol %, from about 1 mol % to about 20 mol %, from about 1 mol % to about 19 mol %, or from about 1 mol % to about 18 mol %, from about 1 mol % to about 17 mol %, from about 1 mol % to about 16 mol %, from about 1 mol % to about 15 mol % or any range therein.

In some embodiments, the proportion of cellulose derivatives or dialdehyde cellulose derivatives or combination thereof present in the lipid nanoparticle compositions is from about 1 mol % to about 25 mol %, from about 2 mol % to about 25 mol %, from about 3 mol % to about 25 mol %, from about 4 mol % to about 25 mol %, from about 2 mol % to about 25 mol %, from about 2 mol % to about 24 mol %, from about 2 mol % to about 23 mol %, from about 2 mol % to about 22 mol %, from about 2 mol % to about 21 mol %, from about 2 mol % to about 20 mol %, from about 2 mol % to about 19 mol %, or from about 2 mol % to about 18 mol %, from about 2 mol % to about 17 mol %, from about 2 mol % to about 16 mol %, from about 2 mol % to about 15 mol % or any range therein.

In some embodiments, the proportion of cellulose derivatives or dialdehyde cellulose derivatives or combination thereof present in the lipid nanoparticle compositions is from about 1 mol % to about 25 mol %, preferably about 1 mol % to about 20 mol %, most preferably about 1 mol % to about 15 mol %.

In some embodiments, the proportion of cellulose or its derivatives or combination thereof present in the lipid nanoparticle compositions is about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, or any portion or fraction thereof.

### Lipid Components

Lipid components of the lipid nanoparticle compositions may include one or more lipids, such as a cationic lipid, a phospholipid, a sterol and a PEG-lipid.

### Cationic Lipid

Cationic lipid refers to a lipid that has a net positive charge at a selected pH. Cationic lipids generally consist of a hydrophilic head group that carries the charge and a hydrophobic tail.

Exemplary cationic lipid for use in the lipid nanoparticle compositions include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)N-2-(sperminecarboxamido)ethyl)-N,N-dimethylammoniumtrifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), N-(1,2-dimyristyloxyprop-3-yl)-N,N-dimethyl-N-hydroxyethyl ammonium bromide (DMRIE), 1,2-dilinoleyloxy-N,N-dimethylaminopropane (DLin-DMA), 3-dimethylamino-2-(cholest-5-en-3-beta-oxybutan-4-oxy)-1-(cis,cis-9,12-oc-tadecadienoxy)propane (Clin-DMA), 2-[5′-(cholest-5-en-3-beta-oxy)-3′-oxapentoxy)-3-dimethyl-1-(cis,cis-9′,12′-octadecadienoxy)propane (CpLin-DMA), 2,3-Dilinoleoyloxy-N,N-dimethylpropylamine (DLin-DAP), 1,2-N,N′-Dilinoleylcarbamyl-3-dimethylaminopropane (DLincarb-DAP), 1,2-Dilinoleoylcarbamyl-3-dimethylaminopropane (DLin-CDAP), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), heptadecan-9-yl 8-[2-hydroxyethyl-(6-oxo-6-undecoxyhexyl)amino]octanoate (SM-102), 6-[6-(2-hexyldecanoyloxy)hexyl-(4-hydroxybutyl)amino]hexyl 2-hexyldecanoate (ALC-0315), nonyl 8-[(8-heptadecan-9-yloxy-8-oxooctyl)-(2-hydroxyethyl)amino]octanoate (SLP-0001) or a combination thereof.

Cationic lipids with amine head group are preferred cationic lipids. The amine group can be at primary, secondary or tertiary position. The cationic lipid may comprise one (monoamine) or more (polyamine) such amine groups.

In some embodiments, the cationic lipids are positively charged at acidic pH i.e., pH 1.0 to pH 6.9. In certain embodiments, the cationic lipids are neutral at certain pH i.e., around physiological pH (pH 7.0 to pH 7.5). A cationic lipid that can exist in a positively charged or neutral form depending on the pH is referred to as ionizable lipid. Preferred cationic lipids are ionizable such that they can exist in a positively charged or neutral form depending on pH. For example, an ionizable lipid may be neutral around physiological pH (pH 7.0 to pH 7.5), and cationic around acidic pH (pH 1.0 to pH 6.9).

In some embodiments, cationic lipid present in the lipid nanoparticle compositions is an ionizable lipid.

Methods of making cationic lipid and/or ionizable lipid or imparting the cationic lipid the ability to behave as an ionizable lipid are well known in the art (WO2005121348; WO2009127060; WO2009086558; WO2010042877; WO2010144740; WO2011075656; WO2017049245; WO2017075531; WO2018118102; WO2015199952; Reynier P. et al. Journal of Drug Targeting (2004) 12: 25-38; Sabnis, Staci et al. Molecular Therapy (2018) 26: 1509-1519).

The proportion of cationic lipid present in the lipid nanoparticle compositions is from about 25 mol % to about 50 mol % or any range therein.

In some embodiments, the proportion of cationic lipid present in the lipid nanoparticle compositions is from about 25 mol % to about 50 mol %, from about 25 mol % to about 48 mol %, from about 25 mol % to about 46 mol %, from about 25 mol % to about 45 mol %, from about 25 mol % to about 44 mol %, from about 25 mol % to about 43 mol %, from about 25 mol % to about 42 mol %, from about 25 mol % to about 41 mol %, from about 25 mol % to about 40 mol %, or any range therein.

In some embodiments, the proportion of cationic lipid present in the lipid nanoparticle compositions is about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 40 mol %, about 41 mol %, about 42 mol %, about 43 mol %, about 44 mol %, about 45 mol %, about 46 mol %, about 47 mol %, about 48 mol %, about 49 mol %, or about 50 mol % or any portion or fraction thereof.

### Phospholipids

Phospholipid includes a lipid containing a hydrophilic head with a phosphate group and a hydrophobic tail composed of fatty acid chains attached to a glycerol or sphingosine backbone.

Exemplary phospholipids for use in the lipid nanoparticle compositions include, but are not limited to, 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), 1-myristoyl-2-stearoyl-sn-glycero-3-phosphocholine (MSPC), 1-palmitoyl-2-myristoyl-sn-glycero-3-phosphocholine (PMPC), 1-palmitoyl-2-stearoyl-sn-glycero-3-phosphocholine (PSPC), 1-stearoyl-2-myristoyl-sn-glycero-3-Phosphocholine (SMPC), 1-Stearoyl-2-palmitoyl-sn-glycero-3-phosphocholine (SPPC), 1-stearoyl-2-oleoyl-sn-glycero-3-phosphocholine (SOPC), 1-stearoyl-2-docosahexaenoyl-sn-glycero-3-phosphocholine (SDPC), sphingomyelin, or combination thereof.

The proportion of phospholipid present in the lipid nanoparticle compositions is from about 2 mol % to about 20 mol % or any range therein.

In some embodiments, the proportion of phospholipid present in the lipid nanoparticle compositions is from about 2 mol % to about 20 mol %, from about 3 mol % to about 19 mol %, from about 3 mol % to about 18 mol %, from about 3 mol % to about 17 mol %, from about 3 mol % to about 16 mol %, from about 3 mol % to about 15 mol %, from about 3 mol % to about 14 mol %, from about 3 mol % to about 13 mol %, from about 3 mol % to about 12 mol %, or any range therein.

In some embodiments, the proportion of phospholipid present in the lipid nanoparticle compositions is about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, or about 20 mol % or any portion or fraction thereof.

Sterol Lipid nanoparticle composition disclosed herein may include sterol and/or sterol derivatives. The term “sterol” as used herein include, but not limited to, cholesterol, sitosterol, fecosterol, ergosterol, campesterol, stigmasterol or their derivatives. In some embodiments, lipid nanoparticle composition comprises cholesterol and/or cholesterol derivatives. Non-limiting examples of cholesterol and cholesterol derivatives include 5α-cholestanol, 5β-coprostanol, cholesteryl-(2′-hydroxy)-ethyl ether, cholesteryl-(4′-hydroxy)-butyl ether, 6-ketocholestanol, 5α-cholestane, cholestenone, 5α-cholestanone, 5β-cholestanone, cholesteryl decanoate, or mixtures thereof. Methods of making cholesterol and cholesterol derivatives are well known in the art (WO2009127060; WO2019152557).

The proportion of sterol present in the lipid nanoparticle compositions may be from about 30 mol % to about 65 mol % or any range therein.

In some embodiments, the proportion of sterol present in the lipid nanoparticle compositions is from about 30 mol % to about 65 mol %, from about 31 mol % to about 60 mol %, from about 32 mol % to about 60 mol %, from about 33 mol % to about 60 mol %, from about 34 mol % to about 60 mol %, from about 35 mol % to about 60 mol %, or any range therein.

In some embodiments, the proportion of sterol present in the lipid nanoparticle compositions is about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, about 65 mol % or any portion or fraction thereof.

### PEG-Lipid

The term PEG-lipid, pegylated lipid, PEG linked lipid, PEG conjugated lipid, PEG-lipid conjugate, PEG modified lipid have been used interchangeably to mean polyethylene glycol linked to a lipid moiety. The lipid moiety may be linked directly to the PEG molecule or through a linker. In some embodiments, a PEG-lipid comprises a PEG-modified phosphatidylethanolamines, PEG-modified phosphatidic acids, PEG-modified ceramides, PEG-modified dialkylamines, PEG-modified diacylglycerols, PEG-modified dialkylglycerols, and/or PEG-modified cholesterol, and/or mixtures thereof. The methods of making PEG-lipid are well known to persons skilled in the art, see for example, US20030077829; US2005008689; U.S. Pat. Nos. 5,885,613; 7,404,969; WO2005026372; WO2009086558).

In some embodiments, PEG-lipid is selected from mPEG-Dimyristoyl glycerol (mPEG-DMG), mPEG-N,N-Ditetradecylacetamide (mPEG-DTA or ALC0159), mPEG-Cholesterol (mPEG-CLS), mPEG-DSPE, mPEG-DMPE, mPEG-DPPE, mPEG-DLPE, mPEG-DOPE, mPEG-DPPC, mPEG-DSPC, 1,2-Distearoyl-sn-Glycero-3-Phosphoethanolamine with conjugated methoxyl poly(ethylene glycol) (mPEG-DSPE), 1,2-dimyristoyl-rac-glycero-3-methoxypolyethylene glycol-2000 (DMG-PEG 2000) or mixtures thereof.

The PEG moiety of the PEG-lipid may comprise an average molecular weight ranging from 0.5 kDa to 10 kDa. In some embodiments, the PEG-lipid has an average molecular weight of about 0.5 kDa to 5 kDa, about 0.5 kDa to 4 kDa, 0.5 kDa to 3 kDa, 0.5 kDa to 2 kDa. In preferred embodiments, the PEG-lipid has an average molecular weight of about 0.5 kDa to about 2 kDa.

The proportion of PEG-lipid present in the lipid nanoparticle compositions may be from about 0.2 mol % to about 2.0 mol % or any range therein.

In some embodiments, the proportion of PEG-lipid present in the lipid nanoparticle compositions is from about 0.2 mol % to about 2.0 mol %, from about 0.2 mol % to about 1.8 mol %, from about 0.2 mol % to about 1.5 mol %, or any range therein.

In some embodiments, the proportion of PEG-lipid present in the lipid nanoparticle compositions is about 0.2 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, or about 2.0 mol % or any portion or fraction thereof.

### Methods of Treatment

Disclosed herein are methods of treating or preventing a disease. The method may comprise administering to a subject in need thereof the lipid nanoparticle composition disclosed herein. The disease may be cancer, an infectious disease or a disease and/or disorder ameliorated by humoral and/or cellular immune response.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

“Humoral immune response” as referred to herein relates to antibody production and the accessory processes that accompany it, such as for example T-helper 2 (Th2) cell activation and cytokine production, isotype switching, affinity maturation and memory cell activation. It also refers to the effector functions of an antibody, such as for example toxin neutralization, classical complement activation, and promotion of phagocytosis and pathogen elimination. The humoral immune response is aided by CD4+Th2 cells and therefore the activation or generation of this cell type is also indicative of a humoral immune response as referred to herein.

A “humoral immune response” as referred to herein may also encompass the generation and/or activation of T-helper 17 (Th17) cells. Th17 cells are a subset of helper effector T-lymphocytes characterized by the secretion of host defense cytokines such as IL-17, IL-17F, IL-21, and IL-22. Th 17 cells are considered developmentally distinct from Th1 and Th2 cells, and have been postulated to facilitate the humoral immune response, such as for example, providing an important function in anti-microbial immunity and protecting against infections. Their production of IL-22 is thought to stimulate epithelial cells to produce antimicrobial proteins and production of IL-17 may be involved in the recruitment, activation and migration of neutrophils to protect against host infection by various bacterial and fungal species.

In some embodiments, the antigen encoded by the nucleic acid in the composition of the disclosure may be a cancer or tumor-associated protein, such as for example, a membrane surface-bound cancer antigen which is capable of being recognized by an antibody.

Cancers that may be treated and/or prevented by the use or administration of a composition of the disclosure include, without limitation, carcinoma, adenocarcinoma, lymphoma, leukemia, sarcoma, blastoma, myeloma, and germ cell tumors. In one embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukemia virus-1. A composition of the disclosure may be used for either the treatment or prophylaxis of cancer, for example, in the reduction of the severity of cancer or the prevention of cancer recurrences. Cancers that may benefit from the compositions of the disclosure include any malignant cell that expresses one or more tumor specific antigens.

In some embodiments, the antigen may be a toxin or an allergen that is capable of being neutralized by an antibody.

In some embodiments, the antigen may be an antigen associated with a disease where it is desirable to sequester the antigen in circulation, such as for example an amyloid protein (e.g., Alzheimer's disease). Thus, a composition of the disclosure may be suitable for use in the treatment and/or prevention of a neurodegenerative disease in a subject in need thereof, wherein the neurodegenerative disease is associated with the expression of an antigen. The subject may have a neurodegenerative disease or may be at risk of developing a neurodegenerative disease. Neurodegenerative diseases that may be treated and/or prevented by the use or administration of a composition of the disclosure include, without limitation, Alzheimer's disease, Parkinson's disease, Huntington's disease, and amyotrophic lateral sclerosis (ALS). For example, Alzheimer's disease is characterized by the association of B-amyloid plaques and/or tau proteins in the brains of patients with Alzheimer's disease (see, for example, Goedert and Spillantini, Science, 314: 777-781, 2006). Herpes simplex virus type 1 has also been proposed to play a causative role in people carrying the susceptible versions of the apoE gene (Itzhaki and Wozniak, J Alzheimers Dis 13: 393-405, 2008).

In some embodiments, the composition may comprise a mixture of B cell epitopes as antigens for inducing a humoral immune response. The B cell epitopes may be linked to form a single polypeptide.

In some embodiments, the antigen may be any peptide or polypeptide that is capable of inducing a specific humoral immune response to a specific conformation on targeted tumor cells.

In some embodiments, the compositions of the present disclosure may be used to induce humoral and/or cellular immune response in a subject. Accordingly, compositions as described herein may be useful for treating or preventing diseases and/or disorders ameliorated by humoral immune responses (e.g., involving B-cells and antibody production). The compositions may find application in any instance in which it is desired to administer an antigen to a subject to induce a humoral immune response or antibody production.

A humoral immune response, as opposed to cell-mediated immunity, is mediated by secreted antibodies which are produced in the cells of the B lymphocyte lineage (B cells). Such secreted antibodies bind to antigens, such as for example those on the surfaces of foreign substances and/or pathogens (e.g., viruses, bacteria, etc.) and flag them for destruction.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigen to T cells, such as T helper CD4 or cytotoxic CD8, thus propagating the immune response. B cells, as well as T cells, are part of the adaptive immune response which is essential for vaccine efficacy. During an active immune response, induced either by vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the toll-like receptors (TLRs).

Antigen presenting cells, such as dendritic cells, macrophages and B cells, are drawn to vaccination sites and can interact with antigens and adjuvants contained in the vaccine. The adjuvant stimulates the cells to become activated and the antigen provides the blueprint for the target. Different types of adjuvants provide different stimulation signals to cells. For example, Poly I:C (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle et al., Curr Med Chem, 2008; So., J Immunol, 2012, which are incorporated hereby by reference in their entireties).

The compositions of the present disclosure, by stimulating strong antibody responses, may be capable of protecting a subject from a disease, disorder or ailment associated with an antigen capable of inducing a humoral immune response.

Without limitation, this includes for example, infectious diseases, cancers involving a membrane surface-bound cancer antigen which is recognized by an antibody, diseases where it is desirable to sequester antigen in circulation, like amyloid protein (e.g., Alzheimer's disease); neutralizing toxins with an antibody; neutralizing viruses or bacteria with an antibody; or neutralizing allergens (e.g., pollen) for the treatment of allergies.

In some embodiments, the composition may be administered via oral, nasal, rectal or parenteral administration. Parenteral administration includes intravenous, intraperitoneal, intradermal, subcutaneous, intramuscular, transepithelial, intrapulmonary, intrathecal, and topical modes of administration. In some embodiments, the composition is administered via intramuscular, subcutaneous or intradermal injection.

The amount of composition used in a single treatment may vary depending on factor such as the nature of negatively charged molecule to be delivered, the type of formulation, and the size of the subject. One skilled in the art will be able to determine, without undue experimentation, the effective amount of composition to use in a particular application.

The skilled artisan can determine suitable treatment regimes, routes of administration, dosages, etc., for any particular application in order to achieve the desired result. Factors that may be taken into account include, e.g., the nature of a polypeptide to be expressed; the disease state to be prevented or treated; the age, physical condition, body weight, sex and diet of the subject; and other clinical factors.

The subject to be treated may be any vertebrate, preferably a mammal, more preferably a human.

### Preparation of Lipid Nanoparticle Composition

Lipid nanoparticles of the composition are typically prepared by mixing a nucleic acid, ionizable polymer and lipid components viz., cationic lipid, phospholipid, sterol and PEG-lipid, not necessarily in the same order.

Lipid nanoparticle compositions can be prepared by mixing the aqueous phase (comprising nucleic acid and ionizable polymer), with organic phase (comprising lipid components i.e., ionizable lipid, phospholipid, sterol and PEG-lipid).

In some embodiments, the pH of the aqueous phase (comprising nucleic acid and ionizable polymer) is from about pH 1.0 to about pH 6.9, from about pH 1.5 to about 6.9, from about 2.0 to about pH 6.9, from about pH 2.5 to about pH 6.9, from about pH 3.0 to about pH 6.9, from about pH 3.5 to about pH 6.9, from about pH 3.8 to about pH 6.9, from about pH 4.0 to about pH 6.9, from about 4.2 to about pH 6.9, from about pH 4.6 to about pH 6.9, from about pH 5.0 to about pH 6.9, from about 6.0 to about pH 6.9 or any range therein.

In some embodiments, the pH of the aqueous phase (comprising nucleic acid and ionizable polymer) is about pH 2.0, about pH 2.1, about pH 2.2, about pH 2.3, about pH 2.4, about pH 2.5, about pH 2.6, about pH 2.7, about pH 2.8, about pH 2.9, about pH 3.0, about pH 3.1, about pH 3.2, about pH 3.3, about pH 3.4, about pH 3.5, about pH 3.6, about pH 3.7, about pH 3.8, about pH 3.9, about pH 4.0, about pH 4.1, about pH 4.2, about pH 4.3, about pH 4.4, about pH 4.5, about pH 4.6, about pH 4.7, about pH 4.8, about pH 4.9, about pH 5.0, about pH 5.1, about pH 5.2, about pH 5.3, about pH 5.4, about pH 5.5, about pH 5.6, about pH 5.7, about pH 5.8, about pH 5.9, about pH 6.0, about pH 6.1, about pH 6.2, about pH 6.3, about pH 6.4, about pH 6.5, about pH 6.6, about pH 6.7, about pH 6.8, about pH 6.9.

In some embodiments, the pH of the aqueous phase (comprising nucleic acid and ionizable polymer) is maintained by a buffer selected from citrate buffer, or acetate buffer.

In some embodiments, aqueous phase (comprising nucleic acide and ionizable polymer), and organic phase (comprising lipid components i.e., ionizable lipid, phospholipid, sterol and PEG-lipid) are mixed using a microfluidic device or jet mixers. Such devices are commercially available from suppliers, for example, The NanoAssemblr devices from Precision Microsystem Inc., Micropore advanced cross flow (AXF) devices from Micropore Technologies, impingement jet mixing skids from Knauer etc. Syringe pumps or pneumatic pressure pumps may be used to inject or deliver the aqueous phase and organic phase to microfluidic device or jet mixers.

In some embodiments, the aqueous phase (comprising nucleic acid and ionizable polymer), and organic phase (comprising lipid components i.e., ionizable lipid, phospholipid, sterol and PEG-lipid) are mixed using a syringe pump in a microfluidic device. The flow rate and flow rate ratio of aqueous phase to organic phase can be appropriately adjusted to enable formation of lipid nanoparticles. The lipid nanoparticles so formed can be subjected to one or more dilution steps, one or more buffer exchange steps and one or more filtration steps.

In some embodiments, lipid nanoparticles formed in accordance with the process are buffer exchanged with phosphate buffered saline so that the final pH of the lipid nanoparticle composition is neutral (pH 7.0 to pH 7.5).

In some embodiments, the ionizable polymer component of the lipid nanoparticle composition is present in the proportion from about from about 1 mol % to about 25 mol %, from about 1 mol % to about 24 mol %, from about 1 mol % to about 23 mol %, from about 1 mol % to about 22 mol %, from about 1 mol % to about 21 mol %, from about 1 mol % to about 20 mol %, from about 1 mol % to about 19 mol %, or from about 1 mol % to about 18 mol %, from about 1 mol % to about 17 mol %, from about 1 mol % to about 16 mol %, from about 1 mol % to about 15 mol % or any range therein.

In some embodiments, the cationic lipid component of the lipid nanoparticle composition is present in the proportion from about 25 mol % to about 50 mol %, from about 25 mol % to about 48 mol %, from about 25 mol % to about 46 mol %, from about 25 mol % to about 45 mol %, from about 25 mol % to about 44 mol %, from about 25 mol % to about 43 mol %, from about 25 mol % to about 42 mol %, from about 25 mol % to about 41 mol %, from about 25 mol % to about 40 mol %, or any range therein.

In some embodiments, the phospholipid component of the lipid nanoparticle composition is present in the proportion of from about 2 mol % to about 20 mol %, from about 3 mol % to about 19 mol %, from about 3 mol % to about 18 mol %, from about 3 mol % to about 17 mol %, from about 3 mol % to about 16 mol %, from about 3 mol % to about 15 mol %, from about 3 mol % to about 14 mol %, from about 3 mol % to about 13 mol %, from about 3 mol % to about 12 mol %, or any range therein.

In some embodiments, the sterol component of the lipid nanoparticle composition is present in the proportion from about 30 mol % to about 65 mol %, from about 31 mol % to about 60 mol %, from about 32 mol % to about 60 mol %, from about 33 mol % to about 60 mol %, from about 34 mol % to about 60 mol %, from about 35 mol % to about 60 mol %, or any range therein.

In some embodiments, the PEG-lipid component of the lipid nanoparticle composition is present in the proportion from about 0.2 mol % to about 2.0 mol %, from about 0.2 mol % to about 1.8 mol %, from about 0.2 mol % to about 1.5 mol %, or any range therein.

In some embodiments, the lipid nanoparticle compositions comprise ionizable polymer from about 1 mol % to about 25 mol %, cationic lipid from about 25 mol % to about 50 mol %, phospholipid from about 2 mol % to about 20 mol %, sterol from about 30 mol % to about 65 mol %, PEG-lipid from about 0.2 mol % to about 2.0 mol %.

The ionizable polymer, cationic lipid, phospholipid, sterol and PEG-lipid are present in mol percentages in the lipid nanoparticle composition such that the sum total of their mol percentage is 100 percent.

In some embodiments, the particle size of the lipid nanoparticle composition may have an average diameter from about 10 nm to about 500 nm, from about 20 nm to about 400 nm, from about 30 nm to about 350 nm, from about 40 nm to about 300 nm, from about 50 nm to about 300 nm, from about 60 nm to about 300 nm or any range therein.

In some embodiments, the particle size of the lipid nanoparticle composition has an average diameter about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 260 nm, about 280 nm, about 290 nm, or about 300 nm.

The lipid nanoparticle composition described herein can be used as a platform for therapeutic or prophylactic delivery of nucleic acids. The nucleic acids may encode one or more antigens, one or more proteins, one or more antibodies or combination thereof. The nucleic acids may regulate or modulate cellular functions. The nucleic acid may belong to any organism such as a prokaryote or a eukaryote, a unicellular organism, a multicellular organism, a virus, a bacterium, a Mycoplasma, a protozoan, an animal or a human.

In some embodiments, the lipid nanoparticle compositions may be used to treat or prevent diseases, such as but not limited to:
diseases caused by viruses belonging to families, for example, picornaviride, calciviridae, astroviridae, togaviridae, flaviviridae, coronoviridae, arteriviridae, rhabndoviridae, filoviridae, paramyxoviridae, bornaviridae, orthomyxoviridae, bunyaviridae, arenaviridae, reoviridae, retroviridae, polyomaviridae, herpesviridae, poxviridae, papillomaviridae, hepadnaviridae, adenoviridae, parvoviridae, hepeviridae, or circoviridae.
diseases caused by bacteria belonging to genera, for example, Bacillus, Bordetella, Borrelia, Brucella, Campylobacter, Chlamydia, Clostridium, Corynebacterium, Enterococcus, Escherichia, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Vibrio, or Yersinia.
cancers, for example, bladder cancer, breast cancer, colon and rectal cancer, endometrial cancer, kidney cancer, leukemia, liver cancer, lung cancer, melanoma, non-hodgkin lymphoma, pancreatic cancer, prostate cancer, or thyroid cancer

The present disclosure is further exemplified by the following non limiting examples. It should be understood that the examples are provided to illustrate the disclosure. From the description and the exemplified embodiments and examples, one skilled in the art can make various modifications or adaptations to the disclosure. Such modifications or adaptations are deemed to be within the scope of the spirit of the disclosure.

### EXAMPLES

Expression of luciferase is one of the standards way for determining the efficiency of nucleic acid based lipid nanoparticle compositions. Accordingly, lipid nanoparticles composition was prepared by using mRNA expressing luciferase. Any other nucleic acid can be used in place of luciferase mRNA.

### Example 1: Preparation of Lipid Nanoparticle Composition/Formulation

Cationic lipid (Octanoic acid, 8-[(2-hydorxyethyl) [8-(nonyloxy)-8-oxooctyl]amino]-, 1-octylnonyl ester—SLP0001), phospholipid (1,2-distearoyl-sn-glycero-3-phosphocholine—DSPC), cholesterol, PEG-lipid (1,2-dimyristoyl-rac-glycero-3-methoxypolytheyleneglycol 2000—DMG-PEG 2000), ionizable polymer (chitosan—low molecular weight) were obtained from Sapala Life Sciences Private Limited, Avanti Polar Lipids, Sigma-Aldrich, Avanti Polar Lipids, and Sigma-Aldrich respectively. Luciferase mRNA was obtained from APExBio (catalog no. R1012).

Different lipid nanoparticle compositions were prepared according to the mol percentages given in table 1.

TABLE 1

| Compositions | | Mol percentage | | | | | |
|---|---|---|---|---|---|---|---|
| or | mRNA | Cationic | | | PEG- | Ionizable | Total mol |
| Formulations | (μg) | lipid | Phospholipid | Cholesterol | lipid | polymer | percentage |
| RF-1 | 100 | 45.05 | 5.03 | 49.62 | 0.31 | — | 100 |
| RF-2 | 100 | 50.0 | 10.0 | 38.5 | 1.5 | — | 100 |
| EF-1 | 100 | 31.9 | 5.5 | 54.2 | 0.4 | 8.0 | 100 |
| EF-2 | 100 | 34.6 | 6.0 | 58.7 | 0.4 | 0.3 | 100 |
| EF-3 | 100 | 32.5 | 5.6 | 55.1 | 0.3 | 6.5 | 100 |
| EF-4 | 100 | 38.5 | 11.5 | 40.5 | 1.5 | 8.0 | 100 |

Note:
RF = reference formulation;
EF = experimental formulation

Nucleic acid (luciferase mRNA) and chitosan were dissolved in aqueous phase (citrate buffer pH 5.2 to 5.6) and lipid components were dissolved in organic phase (100% ethanol). The aqueous phase (continuous) and organic phase (dispersed) were mixed using a dual syringe pump (Microlab 600 dual syringe pump, Hamilton Company) in a microfluidic device (Micropore AXF Mini, Micropore Technologies). The total flow rate was maintained at 30 m/min or 60 m/min, and a flow rate ratio (FRR) of 3:1 for aqueous phase to organic phase respectively. The lipid nanoparticles were diluted with phosphate buffered saline (PBS), pH 7.4, in a ratio of 1:1. The lipid nanoparticles were then buffer exchanged with PBS using 100 kDa filters, and subjected to ultrafiltration step(s) using 0.45 and/or 0.22 micron filters.

### Example 2: Translational Efficacy of Lipid Nanoparticle Composition by Luciferase Assay

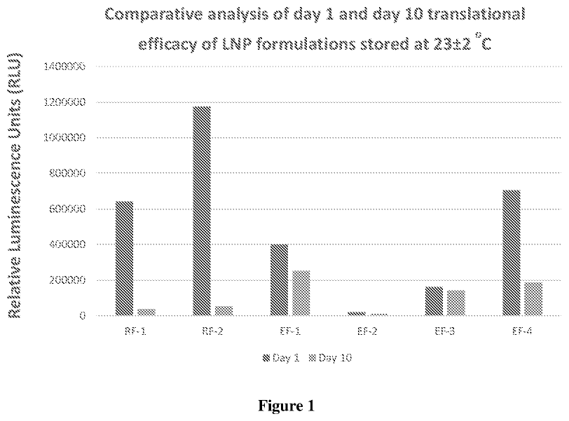

The translational efficacy of lipid nanoparticle composition was estimated by expression of luciferase by luciferase assay. HEK 293 T cells in RPMI medium (supplemented with 10% FBS and 100 units of pen-strep) were seeded in a 12-well plate at a density of 1×10⁵ cells per well. 10 μl of luciferase mRNA encapsulated lipid nanoparticle (luciferase LNP-mRNA) was added to each well. The plates were incubated at 37° C. with 5% CO₂ for 24 hours. After incubation cells were lysed with lysis buffer (1×PBS with 1% NP-40) and centrifuged at 8000 rpm for 10 min at 4° C. The supernatant was collected. 20 μl of supernatant was added to a well of black flat bottom 96-well plate and 100 μl of substrate (Promega Luciferase assay kit Catalogue No. E4030) was added. Immediately after adding substrate luminescence was measured using microplate reader (Infinite 200pro, Tecan). Results are shown in FIG. 1 which indicates drastic reduction in translational efficacy of reference formulations compared to experimental formulations.

### INCORPORATION BY REFERENCE

Each of the patents, published patent applications, and non-patent references cited herein are hereby incorporated by reference in their entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A lipid nanoparticle composition comprising:
a. a nucleic acid,
b. an ionizable polymer,
c. a cationic lipid,
d. a phospholipid,
e. a sterol, and
f. a PEG-lipid.

2. The lipid nanoparticle composition according to claim 1, wherein the nucleic acid is a DNA, an RNA or combination thereof.

3. The lipid nanoparticle composition according to claim 2, wherein the RNA is a messenger RNA (mRNA), a non-coding RNA (ncRNA) or combination thereof.

4. The lipid nanoparticle composition according to claim 3, wherein the non-coding RNA (ncRNA) is long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA) or ribosomal RNA (rRNA) or combination thereof.

5. The lipid nanoparticle composition according to any one of the preceding claims, wherein the ionizable polymer is present in an amount from 1 mol percent to 25 mol percent.

6. The lipid nanoparticle composition according to any one of the preceding claims 1-5, wherein the ionizable polymer comprises a biocompatible polymer.

7. The lipid nanoparticle composition according to any one of the preceding claims 1-6, wherein the ionizable polymer is a chitosan, a cellulose derivative, a poly-L-lysine, a poly-L-glutamic acid, and/or their derivatives or combination thereof.

8. The lipid nanoparticle composition according to claim 7, wherein the ionizable polymer is a chitosan, a cellulose derivative, and/or their derivatives or combination thereof.

9. The lipid nanoparticle composition according to claim 8, wherein the chitosan, chitosan derivatives or combination thereof is present in an amount from 1 mol percent to 25 mol percent.

10. The lipid nanoparticle composition according to claim 8, wherein the cellulose derivative is present in an amount from 1 mol percent to 25 mol percent.

11. The lipid nanoparticle composition according to claim 9, wherein the chitosan or its derivatives or combination thereof is present in an amount from 1 mol percent to 25 mol percent, 1 mol percent to 20 mol percent, or 1 mol percent to 15 mol percent.

12. The lipid nanoparticle composition according to any one of the preceding claims, wherein the cationic lipid is present in an amount from 25 mol percent to 50 mol percent.

13. The lipid nanoparticle composition according to any one of the preceding claims, wherein the cationic lipid is N,N-dioleyl-N,Ndimethylammonium chloride (DODAC); N-(2,3-dioleyloxy)propyl)-N,N,Ntrimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammoniumtrifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), N-(1,2-dimyristyloxyprop-3-yl)-N,N-dimethyl-N-hydroxyethyl ammonium bromide (DMRIE), 1,2-dilinoleyloxy-N,N-dimethylaminopropane (DLin-DMA), 3-dimethylamino-2-(cholest-5-en-3-beta-oxybutan-4-oxy)-1-(cis,cis-9,12-oc-tadecadienoxy)propane (Clin-DMA), 2-[5′-(cholest-5-en-3-beta-oxy)-3′-oxapentoxy)-3-dimethyl-1-(cis,cis-9′,12′-octadecadienoxy)propane (CpLin-DMA), 2,3-Dilinoleoyloxy-N,N-dimethylpropylamine (DLin-DAP), 1,2-N,N′-Dilinoleylcarbamyl-3-dimethylaminopropane (DLincarb-DAP), 1,2-Dilinoleoylcarbamyl-3-dimethylaminopropane (DLin-CDAP), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), heptadecan-9-yl 8-[2-hydroxyethyl-(6-oxo-6-undecoxyhexyl)amino]octanoate (SM-102), 6-[6-(2-hexyldecanoyloxy)hexyl-(4-hydroxybutyl)amino]hexyl 2-hexyldecanoate (ALC-0315), and nonyl 8-[(8-heptadecan-9-yloxy-8-oxooctyl)-(2-hydroxyethyl)amino]octanoate (SLP-0001).

14. The lipid nanoparticle composition according to any one of the preceding claims, wherein the cationic lipid comprises an ionizable lipid.

15. The lipid nanoparticle composition according to claim 14, wherein the ionizable lipid is present in an amount from 25 mol percent to 50 mol percent.

16. The lipid nanoparticle composition according to any one of the preceding claims, wherein the phospholipid is present in an amount from 2 mol percent to about 20 mol percent.

17. The lipid nanoparticle composition according to claim 16, wherein the phospholipid is 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), 1-myristoyl-2-stearoyl-sn-glycero-3-phosphocholine (MSPC), 1-palmitoyl-2-myristoyl-sn-glycero-3-phosphocholine (PMPC), 1-palmitoyl-2-stearoyl-sn-glycero-3-phosphocholine (PSPC), 1-stearoyl-2-myristoyl-sn-glycero-3-Phosphocholine (SMPC), 1-Stearoyl-2-palmitoyl-sn-glycero-3-phosphocholine (SPPC), 1-stearoyl-2-oleoyl-sn-glycero-3-phosphocholine (SOPC), 1-stearoyl-2-docosahexaenoyl-sn-glycero-3-phosphocholine (SDPC), sphingomyelin, and combination thereof.

18. The lipid nanoparticle composition according to any one of the preceding claims, wherein the sterol is present in an amount from 30 mol percent to about 65 mol percent.

19. The lipid nanoparticle composition according to any one of the preceding claims, wherein the sterol is cholesterol, sitosterol, fecosterol, ergosterol, campesterol, stigmasterol, 5α-cholestanol, 5β-coprostanol, cholesteryl-(2′-hydroxy)-ethyl ether, cholesteryl-(4′-hydroxy)-butyl ether, 6-ketocholestanol, 5α-cholestane, cholestenone, 5α-cholestanone, 5β-cholestanone, cholesteryl decanoate, or their derivatives.

20. The lipid nanoparticle composition according to any one of the preceding claims, wherein the PEG-lipid is present in an amount from 0.2 mol percent to about 2.0 mol percent.

21. The lipid nanoparticle composition according to any one of the preceding claims, wherein the PEG-lipid is mPEG-Dimyristoyl glycerol (mPEG-DMG), mPEG-N,N-Ditetradecylacetamide (mPEG-DTA or ALC0159), mPEG-Cholesterol (mPEG-CLS), mPEG-DSPE, mPEG-DMPE, mPEG-DPPE, mPEG-DLPE, mPEG-DOPE, mPEG-DPPC, mPEG-DSPC, 1,2-Distearoyl-sn-Glycero-3-Phosphoethanolamine with conjugated methoxyl poly(ethylene glycol) (mPEG-DSPE), 1,2-dimyristoyl-rac-glycero-3-methoxypolyethylene glycol-2000 (DMG-PEG 2000), or mixtures thereof.

22. The lipid nanoparticle composition according to any one of the preceding claims, wherein the nucleic acid encodes an antigenic polypeptide or regulate or module a cellular function.

23. The lipid nanoparticle composition according claim 22, wherein the antigenic polypeptide is derived from an infectious agent.

24. The lipid nanoparticle composition according claim 23, wherein the infectious agent is selected from strains of viruses and strains of bacteria.

25. The lipid nanoparticle composition according to any one of the preceding claims, wherein the nucleic acid comprise at least one chemical modification.

26. A nucleic acid vaccine comprising the lipid nanoparticle composition according to any one of the preceding claims.

27. A method of treating or preventing a disease comprising administering to a subject in need thereof the lipid nanoparticle composition according to any one of claims 1-25 or the nucleic acid vaccine according to claim 26.

28. The method of claim 27, wherein the disease is cancer, an infectious disease or a disease and/or disorder ameliorated by humoral and/or cellular immune response.

29. Use of the lipid nanoparticle composition according to any one of the preceding claims 1-25 in the manufacture of a medicament for the treatment of a disease in a subject.

30. The use of claim 29, wherein the disease is cancer, an infectious disease or a disease and/or disorder ameliorated by humoral and/or cellular immune response.

31. A method of preparing the lipid nanoparticle composition of any one of the preceding claims 1-25 comprising mixing an aqueous phase comprising the nucleic acid and the ionizable polymer, and an organic phase comprising the cationic lipid, the phospholipid, the cholesterol and the PEG-lipid.
